(19) United States
(12) Patent Application Publication
(10) Pub. No.: US 2023/0172721 A1
(43) Pub. Date: Jun. 8, 2023

(54) SPINAL IMPLANT DEVICE

(71) Applicant: Ali ANISSIPOUR, Everett, WA (US)

(72) Inventor: Ali ANISSIPOUR, Everett, WA (US)

(21) Appl. No.: 18/075,204

(22) Filed: Dec. 5, 2022

Related U.S. Application Data
(60) Provisional application No. 63/287,168, filed on Dec. 8, 2021.

(51) Int. Cl. A61F 2/44; A61F 2/30; A61F 2/46

(52) U.S. Cl. CPC A61F 2/4455; A61F 2/4611; A61F 2/30749; A61F 2002/2835; A61F 2002/3054; A61F 2002/30579

(57) ABSTRACT

An example spinal device including an upper plate, a lower plate, at least one front link pivotably coupled to a front portion of the upper plate and pivotably coupled to a front portion of the lower plate, and at least one rear link pivotably coupled to a rear portion of the upper plate and pivotably coupled to a rear portion of the lower plate. The spinal device is selectively movable from a collapsed configuration to an expanded configuration. The spinal device is moved from the collapsed configuration to the expanded configuration by moving the upper plate relative to the lower plate, the moving of the upper plate relative to the lower plate including pivoting of the front link and the rear link relative to the upper plate and the lower plate, the pivoting of the front link and the rear link causing vertical separation of the upper plate relative to the lower plate.

This application claims the benefit of U.S. Provisional Pat. Application No. 63/287,168, filed Dec. 8, 2021, which is incorporated by reference herein in its entirety.

### BACKGROUND

Spinal devices and implants are used to treat various spinal conditions in patients. For example, for patients with spondylolisthesis, a spinal implant may be used to reposition vertebrae. Some such implants are useful for expanding the disc space between vertebrae. Lateral interbody fusions surgeries are indicated to treat spondylolisthesis. Correction from a lateral approach is aimed at correction of height and lordosis. The ability to reduce a spondylolisthesis from the lateral approach is limited and largely reliant on ligamentotaxis.

### BRIEF DESCRIPTION OF THE DRAWINGS

For a more complete understanding of various examples, reference is now made to the following descriptions taken in connection with the accompanying drawings in which:

FIG. 1 is a perspective view of an example spinal device;

FIG. 2 is a frontal view of the example spinal device of FIG. 1;

FIG. 3 is a top view of the example spinal device of FIGS. 1 and 2;

FIG. 4 is a perspective view of the example spinal device of FIGS. 1-3 with attachment screws;

FIG. 5 is a side view of the example spinal device of FIG. 4;

FIG. 6 is a perspective view of an example expansion tool for use with the example spinal device of FIGS. 1-5;

FIG. 7 is a perspective view of the example spinal device of FIGS. 1-5 with the example expansion tool of FIG. 6 prior to expansion of the example spinal device;

FIG. 8 is a perspective view of the example spinal device of FIGS. 1-5 with the example expansion tool of FIG. 7 after expansion of the example spinal device,

FIG. 9 is a side view of the example spinal device of FIGS. 1-5 with the example expansion tool of FIG. 6 prior to expansion of the example spinal device;

FIG. 10 is a side view of the example spinal device of FIGS. 1-5 with the example expansion tool of FIG. 6 after expansion of the example spinal device;

FIG. 11 is a side view of the example spinal device of FIGS. 1-5 attached to vertebrae with the example expansion tool of FIG. 6 prior to expansion of the example spinal device;

FIG. 12 is a side view of the example spinal device of FIGS. 1-5 attached to vertebrae with the example expansion tool of FIG. 6 after expansion of the example spinal device;

### DETAILED DESCRIPTION

Various examples described herein provide spinal devices or implants and methods to treat various spinal conditions such as spondylolisthesis. The example spinal devices described herein may be used in a lateral procedure to regain height and lordosis while simultaneously reducing the spondylolisthesis by causing a posterior translation of the cephalad vertebra relative to the subjacent vertebrae. Further, the example spinal devices may be used to facilitate intervertebral fusion.

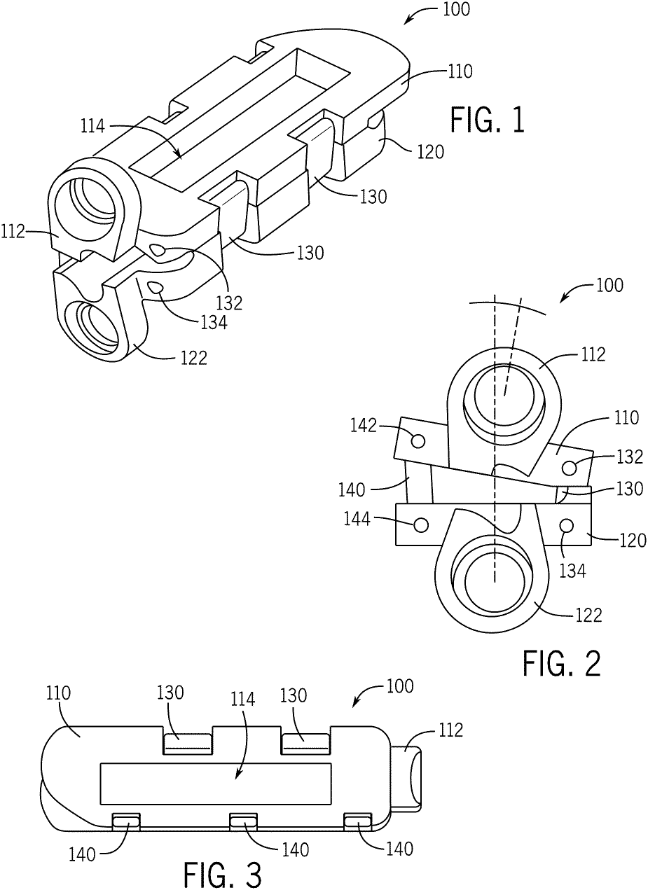

Referring now to the Figures, FIG. 1 is a perspective view of an example spinal device 100. FIG. 2 is a frontal view of the example spinal device 100, and FIG. 3 is a top view of the example spinal device 100. In various examples, the spinal device 100 is an intervertebral fusion device. In this regard, the example spinal device 100 is intended to be positioned between two vertebrae and to expand a disc space between the two vertebrae. A graft may be positioned within the example spinal device 100 between the two vertebrae to facilitate fusion of the two vertebrae. The example spinal device 100 is selectively movable from a collapsed configuration to an expanded configuration. In this regard, the example spinal device 100 can be inserted between two vertebrae in a collapsed configuration and moved to an expanded configuration to expand the disc space between the two vertebrae.

The example spinal device 100 includes an upper plate 110 and a lower plate 120. The upper plate 110 is sized to engage an upper vertebra. In this regard, when the example spinal device 100 is inserted between two vertebrae, the upper surface of the upper plate 110 is in contact with a bottom surface of the upper vertebra.

Similarly, the lower plate 120 is sized to engage a top surface of a lower vertebra. Thus, when the example spinal device 100 is inserted between the two vertebrae, the lower surface of the lower plate 120 is in contact with the top surface of the lower vertebra.

The upper plate 110 includes an upper plate screw insertion portion 112 to allow the upper plate to be secured to the upper vertebra. Similarly, the lower plate 120 includes a lower plate screw insertion portion 122 to allow the lower plate to be secured to the lower vertebra. In the example spinal device 100, the upper plate screw insertion portion 112 and the lower plate screw insertion portion 122 are provided on a left side of the upper plate 110 and the lower plate 120, respectively. In this regard, the example spinal device 100 is configured for lateral insertion from the left side of the body. As described below with reference to FIGS. 11 and 12, screws may be inserted through the upper plate screw insertion portion 112 and the lower plate screw insertion portion 122 to secure the upper plate 110 and the lower plate 120 to the upper vertebra and the lower vertebra, respectively.

The upper plate 110 is provided with an upper fusion graft cavity 114 to allow insertion of a bone graft therein. The lower plate 120 is similarly provided with a lower fusion graft cavity (not shown in the Figures). With the example spinal device 100 in an expanded configuration, a bone graft can be positioned in the graft cavity. When inserted, the bone graft facilitates fusion of the upper vertebra and the lower vertebra.

A rear portion of the upper plate 110 is pivotably coupled to one end of at least one rear link 130. The upper plate 110 is pivotable relative to the rear link 130 around an upper rear connector pin 132. The other end of the at least one rear link 130 is pivotably coupled to a rear portion of the lower plate 120. The lower plate 120 is pivotable relative to the rear link 130 around a lower rear connector pin 134. Thus, at least one rear link 130 couples the rear portion of the upper plate 110 to the rear portion of the lower plate 120. In the example spinal device 100, two rear links 130 are provided. In other examples, a different number of rear links 130 may be provided.

A front portion of the upper plate 110 is pivotably coupled to one end of at least one front link 140. The upper plate 110 is pivotable relative to the front link 140 around an upper front connector pin 142. The other end of the at least one front link 140 is pivotably coupled to a front portion of the lower plate 120. The lower plate 120 is pivotable relative to the front link 140 around a lower front connector pin 144. Thus, the at least one front link 140 couples the front portion of the upper plate 110 to the front portion of the lower plate 120. In the example spinal device 100, two front links 140 are provided. In other examples, a different number of front links 140 may be provided.

In the example spinal device 100 illustrated in FIGS. 1-3, the front links 140 are longer than the rear links 130. Thus, with the spinal device in the expanded configuration (as most clearly illustrated in FIG. 2), the upper plate 110 and the lower plate 120 are not parallel to each other, but rather have a non-zero angle between the two. In one example, the angle is between 5 degrees and 15 degrees, and more preferably about 10 degrees. When inserted between vertebrae and moved in to the expanded configuration, the angle provides increasing lordosis.

As used herein, terms such as “upper”, “lower”, “front”, “rear,” “left” and “right” refer to direction or orientation within the human body. For example, the upper plate 110 is closer to the upper end of the human body (the head), while the lower plate 120 is positioned closer to the lower end of the body (the feet). Similarly, front link 140 is positioned towards the front of the body relative to the rear link 130, which is positioned towards the rear of the body.

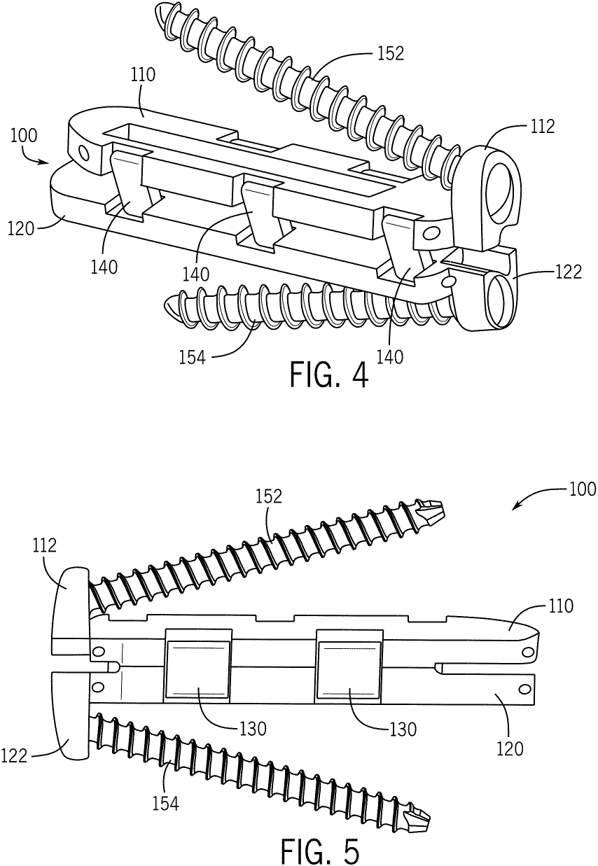

Referring now to FIGS. 4 and 5, FIG. 4 is a perspective view of the example spinal device 100 of FIGS. 1-3 with attachment screws, and FIG. 5 is a side view of the arrangement of FIG. 4. As described above, the upper plate 110 and lower plate 120 are provided with upper plate screw insertion portion 112 and a lower plate screw portion 122, respectively. As illustrated in FIGS. 4 and 5, an upper attachment screw 152 is inserted through the upper plate screws insertion portion 112. The upper attachment screw 152 is screwed into the upper vertebra to secure the upper plate to the upper vertebra. Similarly, a lower attachment screw 154 is inserted through the lower plate screw insertion portion 122. The lower attachment screw 154 is screwed into the lower vertebra to secure the lower plate to the lower vertebra.

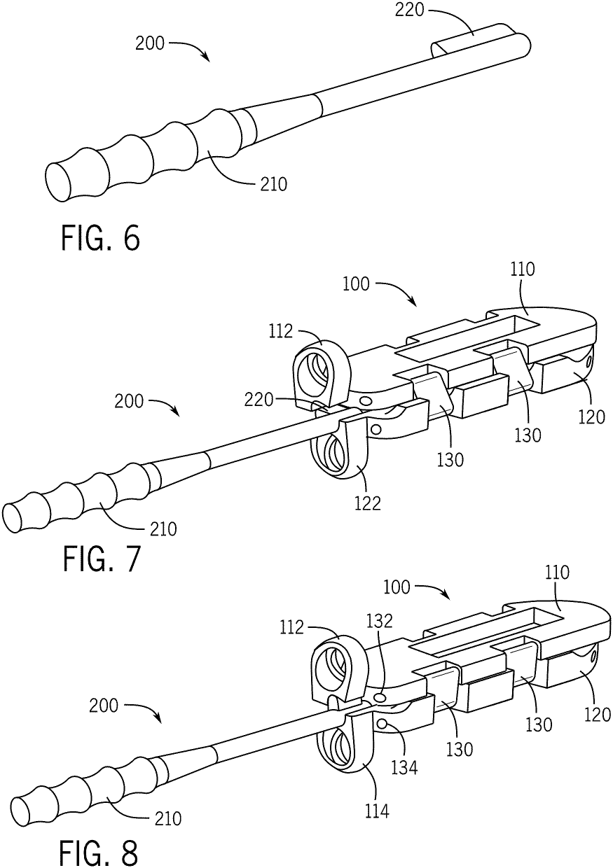

FIG. 6 is a perspective view of an example expansion tool 200 for use with the example spinal device of FIGS. 1-5. The expansion tool 200 includes a handle 210 to allow an operator to grasp and control the expansion tool 200. The expansion tool 200 includes an engagement portion 220 to engage the example spinal device 100. The expansion tool 200 is used to move the example spinal device 100 from a collapsed configuration to an expanded configuration, as described below.

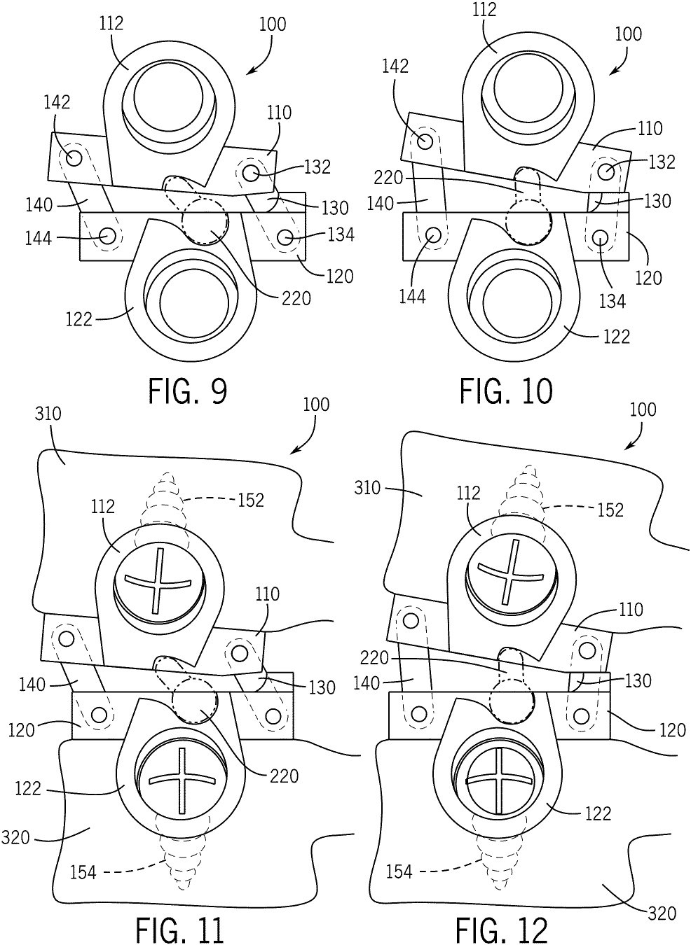

FIG. 7 is a perspective view of the example spinal device 100 with the example expansion tool 200 prior to expansion of the example device, and FIG. 8 is a perspective view of the example spinal device 100 with the example expansion tool 200 prior after expansion of the example device. FIG. 9 is a side view of the arrangement of FIG. 7 prior to expansion of the example spinal device 100, and FIG. 10 is a side view of the arrangement of FIG. 8 after expansion of the example spinal device 100.

As noted above, the example spinal device 100 is selectively movable from a collapsed configuration to an expanded configuration. FIGS. 7 and 9 illustrate the example spinal device 100 in the collapsed configuration. In FIGS. 7-10, the expansion tool 200 is shown inserted into the example spinal device 100. In the collapsed configuration, the engagement portion 220 of the expansion tool 200 is positioned flat and parallel to the upper plate 110 and the lower plate 120. From the collapsed configuration shown in FIGS. 7 and 9, the expansion tool 200 is rotated to move the example spinal device 100 from the collapsed configuration to the expanded configuration. In the expanded configuration shown in FIGS. 8 and 10, the engagement portion 220 of the expansion tool 200 is positioned substantially vertically and substantially perpendicular to the upper plate 110 and the lower plate 120. The example spinal device 100 is thus moved from the collapsed configuration to the expanded configuration by moving the upper plate 110 relative to the lower plate 120. Moving the upper plate 110 relative to the lower plate 120 includes pivoting of the front links 140 and the rear links 130 relative to the upper plate 110 and the lower plate 120. Pivoting of the front links 140 and the rear links 130 causes vertical separation of the upper plate 110 relative to the lower plate 120, resulting in expansion of the space (or disc space) between the upper plate 110 and the lower plate 120. Additionally. The pivoting of the front links 140 and the rear links 130 causes posterior translation of the upper plate 110 relative to the lower plate 120.

In various examples, the example spinal device 100 includes a locking feature which locks the example spinal device in the expanded configuration. In the example illustrated in FIGS. 7-10, the locking feature is in the form of the positions of the front links 140 and the rear links 130. In particular, as illustrated most clearly in FIG. 9, in the collapsed configuration, the rear links 130 and the front links 140 are slanted in the same direction as each other. In particular, FIG. 9 illustrates both the front links 140 and the rear links 130 slanted leftward relative to a vertical plane.

In the expanded configuration most clearly shown in FIG. 10, the rear links 130 and the front links 130 are slanted in opposite directions from the vertical plant from each other. In particular, FIG. 10 illustrates the front links 140 are slanted leftward relative to a vertical plane, while the rear links 130 are slanted rightward relative to the vertical plane. Thus, the front links 140 and the rear links 130 form a “V” shape. With the pressure supplied between the upper vertebra and the lower vertebra, the example spinal device 130 is substantially locked in the expanded configuration.

Other examples of the spinal device 100 may be provided with other forms of locking mechanism. For example, the spinal device 100 can be provided with a lock screw mechanism which mechanically secures the spinal device 100 in the expanded configuration.

As noted above, the example spinal device 100 can restore lordosis. In this regard, as most clearly illustrated in FIG. 10, in the expanded configuration, the vertical separation of the upper plate 110 and the lower plate 120 includes larger separation at the front portions of the upper plate 110 and the lower plate 120 and a smaller separation at the rear portions of the upper plate 110 and the lower plate 120. This differential separation results from the sizing of the front links 140 and the rear links 130. By making the front links 140 longer than the rear links 130, the larger separation at the front portions and a smaller separation at the rear portions is achieved.

Referring now to FIGS. 11 and 12, side views of the example spinal device 100 illustrate the example spinal device 100 attached to vertebrae prior to and after, respectively, expansion of the example spinal device 100. As illustrated in FIG. 11, the example spinal device 100 is positioned in the collapsed configuration between an upper vertebra 310 and a lower vertebra 320. The upper plate 110 of the example spinal device 10 is in physical communication with the upper vertebra 310, and the lower plate 120 is in physical communication with the lower vertebra 320.

Referring now to FIG. 12, the example spinal device 100 is reconfigured from the collapsed configuration of FIG. 11 to an expanded configuration. As noted above, the reconfiguring to the expanded configuration is achieved by moving the upper plate 110 relative to the lower plate 120, including pivoting of the front links 140 and the rear links 130 relative to the upper plate 110 and the lower plate 120. The pivoting of the front links 140 and the rear links 130 cause vertical separation of the upper plate 110 relative to the lower plate 120. As also noted above, pivoting of the front links 140 and the rear links 130 causes posterior translation of the upper plate 110 relative to the lower plate 120.

The example spinal device 110 is secured in place by securing the upper plate 110 to the upper vertebra 310, and securing the lower plate 120 to the lower vertebra 320. The securing of the upper plate 110 and the lower plate 120 is achieved by inserting screws 152, 154 through the screw insertion portions 112, 122 of the upper plate 110 and the lower plate 120 and into the corresponding vertebra 310, 320. In one example, as illustrated in FIG. 9, the upper plate 110 and the lower plate 120 are secured to the corresponding vertebra 310, 320 while the spinal device 100 is still in the collapsed configuration, prior to reconfiguring the example spinal device 100 to an expanded configuration.

In other examples, the securing of the example spinal device 100 to the vertebrae is performed after the example spinal device 100 is reconfigured into the expanded configuration. In this regard, the spinal device 100 is inserted between the vertebrae in the collapsed configuration, moved to the expanded configuration, and then secured to the vertebrae.

In various examples, with the spinal device 100 in the expanded configuration, the fusion graft cavity 114 of the upper plate 110 and a corresponding fusion graft cavity of the lower plate together form an intervertebral cavity. A bone graft can be inserted into the intervertebral cavity to facilitate fusion of the upper vertebra 310 and the lower vertebra 320.

In various examples, the example spinal device 100 is inserted between the upper vertebra 310 and the lower vertebra 320 with a lateral approach. For example, a lateral retroperitoneal approach may be used. The example spinal device 100 may be inserted transpsoas or antepsoas. Further, with the example spinal device 100 in the expanded configuration, a bone graft may be inserted with the same approach. For example, the bone graft may be inserted into the above-described intervertebral cavity through a space between the screw insertion portions 112, 122. The upper plate fusion graft cavity 114 and a corresponding cavity in the lower plate 120 allow for the fusion of the vertebrae 310, 320.

Thus, various examples of the spinal device can simultaneously provide (1) expansion of the disc space between vertebrae, (2) restoration of lordosis, and (3) reduction of the forward slippage of the vertebrae. For example, in the expanded configuration, the example spinal device 100 provides expansion of the disc space by providing vertical separation between the lower vertebra 120 and upper vertebra 120. Further, as described above with reference to FIG. 10 above, the sizing of the front links 140 as larger than the rear links 130 results in the larger separation at the front portions of the upper plate 110 and the lower plate 120 and the smaller separation at the rear portions of the upper plate 110 and the lower plate 120. This differential in height between the front portions and the rear portions of the upper plate 1 10 and the lower plate 120 serves to restore lordosis. Finally, with the example spinal device 100 locked in the expanded configuration, combined with securing of the upper plate 110 to the upper vertebra 310 and securing of the lower plate 120 to the lower vertebra 320, the example spinal device 100 results in a posterior translation of the upper vertebra 310 relative to the lower vertebra 320.

The foregoing description of various examples has been presented for purposes of illustration and description. The foregoing description is not intended to be exhaustive or limiting to the examples disclosed, and modifications and variations are possible in light of the above teachings or may be acquired from practice of various examples. The examples discussed herein were chosen and described in order to explain the principles and the nature of various examples of the present disclosure and its practical application to enable one skilled in the art to utilize the present disclosure in various examples and with various modifications as are suited to the particular use contemplated. The features of the examples described herein may be combined in all possible combinations of methods, apparatus, modules, systems, and computer program products.

It is also noted herein that while the above describes examples, these descriptions should not be viewed in a limiting sense. Rather, there are several variations and modifications which may be made without departing from the scope as defined in the appended claims.

What is claimed is:

1. A spinal device, comprising:
an upper plate;
a lower plate;
at least one front link pivotably coupled to a front portion of the upper plate and pivotably coupled to a front portion of the lower plate; and
at least one rear link pivotably coupled to a rear portion of the upper plate and pivotably coupled to a rear portion of the lower plate,
wherein the spinal device is selectively movable from a collapsed configuration to an expanded configuration;
wherein the spinal device is moved from the collapsed configuration to the expanded configuration by moving the upper plate relative to the lower plate, the moving of the upper plate relative to the lower plate including pivoting of the front link and the rear link relative to the upper plate and the lower plate, the pivoting of the front link and the rear link causing vertical separation and posterior translation of the upper plate relative to the lower plate.

2. The spinal device of claim 1, wherein:
in the collapsed configuration, the front link and the rear link are slanted in the same direction from a vertical plane as each other, and
in the expanded configuration, the front link and the rear link are slanted in opposite directions from a vertical plane from each other.

3. The spinal device of claim 1, wherein, in the front link is longer than the rear link.

4. The spinal device of claim 3, wherein, in the expanded configuration, the vertical separation of the upper plate and the lower plate includes larger separation at the front portions of the upper plate and the lower plate and a smaller separation at the rear portions of the upper plate and the lower plate.

5. The spinal device of claim 1, wherein each of the upper plate and the lower plate include a screw insertion portion to facilitate securing the upper plate and the lower plate to a corresponding vertebra.

6. The spinal device of claim 5, wherein the screw insertion portions of the upper plate and the lower plate facilitate securing of the upper plate and the lower plate to a lateral portion of the corresponding vertebra.

7. The spinal device of claim 6, wherein, in the expanded configuration and with upper plate and the lower plate secured to the lateral portion of the corresponding vertebra, the spinal device provides (1) expansion of the disc space between vertebrae, (2) restoration of lordosis, and (3) reduction of slipping of the vertebrae.

8. The spinal device of claim 1, wherein the upper plate and the lower plate each include a fusion graft cavity to receive a bone graft therein.

9. The spinal device of claim 8, wherein, in the expanded configuration, the fusion graft cavity of the upper plate and the fusion graft cavity together form a intervertebral cavity to receive a bone graft to fuse adjacent vertebrae.

10. A method, comprising:
positioning a spinal device in a collapsed configuration between an upper vertebra and a lower vertebra, the spinal device including:
an upper plate in physical communication with the upper vertebra;
a lower plate in physical communication with the lower vertebra;
at least one front link pivotably coupled to a front portion of the upper plate and pivotably coupled to a front portion of the lower plate; and
at least one rear link pivotably coupled to a rear portion of the upper plate and pivotably coupled to a rear portion of the lower plate;
reconfiguring the spinal device from the collapsed configuration to an expanded configuration by moving the upper plate relative to the lower plate, the moving of the upper plate relative to the lower plate including pivoting of the front link and the rear link relative to the upper plate and the lower plate, the pivoting of the front link and the rear link causing vertical separation of the upper plate relative to the lower plate.

11. The method of claim 10, further comprising:
securing the upper plate to the upper vertebra; and
securing the lower plate to the lower vertebra.

12. The method of claim 11, wherein, in the expanded configuration and with upper plate secured to the upper vertebra and the lower plate secured to the lower vertebra, the spinal device provides (1) expansion of the disc space between vertebrae, (2) restoration of lordosis, and (3) posterior translation of the superior vertebrae resulting in reduction of slipping of the vertebrae.

13. The method of claim 11, wherein the securing of upper plate to the upper vertebra and the securing of the lower plate to the lower vertebra are performed prior to the reconfiguring of the spinal device.

14. The method of claim 11, wherein the securing of upper plate to the upper vertebra and the securing of the lower plate to the lower vertebra are performed after the reconfiguring of the spinal device.

15. The method of claim 11,
wherein the securing the upper plate to the upper vertebra includes inserting a fastener through a screw insertion portion of the upper plate and into the upper vertebra; and
wherein the securing the lower plate to the lower vertebra includes inserting a fastener through a screw insertion portion of the lower plate and into the lower vertebra.

16. The method of claim 15, wherein the fasteners are inserted into a lateral portion of the corresponding vertebra.

17. The method of claim 10, wherein the upper plate and the lower plate each include a fusion graft cavity, the fusion graft cavity of the upper plate and the fusion graft cavity of the lower plate together forming an intervertebral cavity.

18. The method of claim 17, further comprising inserting a bone graft into the intervertebral cavity to facilitate fusion of the upper vertebra and the lower vertebra.

19. The method of claim 10, wherein reconfiguring the spinal device comprises:
inserting a tool between the upper plate and the lower plate;
and rotating the tool to cause the upper plate and lower plate to move relative to each other;
removing the tool when the spinal device has been reconfigured to the expanded configuration.

20. The method of claim 10, wherein
in the collapsed configuration, the front link and the rear link are slanted in the same direction from a vertical plane as each other, and
in the expanded configuration, the front link and the rear link are slanted in opposite directions from a vertical plane from each other.

21. The method of claim 20, wherein reconfiguring the spinal device comprises:
inserting a tool between the upper plate and the lower plate;
rotating the tool to cause the upper plate and lower plate to move relative to each other; and
removing the tool when the front link and the rear link are slanted in opposite directions from the vertical plane from each other.
